(19) United States
(12) Patent Application Publication
(10) Pub. No.: US 2004/0092566 A1
(43) Pub. Date: May 13, 2004

(54) Celecoxib prodrug

(76) Inventors: Matthew J. Graneto, Chesterfield, MO (US); Gary D. Ewing, Kalamazoo, MI (US)

Correspondence Address: PHARMACIA CORPORATION, Global Patent Department, 575 Maryville Centre Drive, Mail Zone 1006, St. Louis, MO, 63141, US

(21) Appl. No.: 10/667,622

(22) Filed: Sep. 22, 2003

Related U.S. Application Data
(60) Provisional application No. 60/425,703, filed on Nov. 12, 2002.

(51) Int. Cl. A61K 31/415; C07D 231/12

(52) U.S. Cl. 514/406; 548/377.1

(57) ABSTRACT

N-[[4-[5-(4-methylphenyl)-3-(trifluoromethyl)-1H-pyrazol-1-yl]phenyl]-sulfonyl]propanamide and pharmaceutically acceptable salts thereof are useful prodrugs of the selective COX-2 inhibitory drug celecoxib, which can be administered to a subject by any suitable route.

This application claims priority of U.S. provisional application Serial No. 60/425,703 filed on Nov. 12, 2002.

### FIELD OF THE INVENTION

The present invention relates to a prodrug of the selective cyclooxygenase-2 (COX-2) inhibitory drug celecoxib.

### BACKGROUND OF THE INVENTION

Inhibition of cyclooxygenase (COX) enzymes is believed to be at least the primary mechanism by which nonsteroidal anti-inflammatory drugs (NSAIDs) exert their characteristic anti-inflammatory, antipyretic and analgesic effects, through inhibition of prostaglandin synthesis. Conventional NSAIDs such as ketorolac, diclofenac, naproxen and salts thereof inhibit both the constitutively expressed COX-1 and the inflammation-associated or inducible COX-2 isoforms of cyclooxygenase at therapeutic doses. Inhibition of COX-1, which produces prostaglandins that are necessary for normal cell function, appears to account for certain adverse side effects that have been associated with use of conventional NSAIDs. By contrast, selective inhibition of COX-2 without substantial inhibition of COX-1 leads to anti-inflammatory, antipyretic, analgesic and other useful therapeutic effects while minimizing or eliminating such adverse side effects. Selective COX-2 inhibitory drugs such as celecoxib and rofecoxib, first commercially available in 1999, have therefore represented a major advance in the art. These drugs are formulated in a variety of orally deliverable dosage forms.

Parecoxib, disclosed in U.S. Pat. No. 5,932,598 to Talley et al., incorporated herein by reference, is one of a class of N-substituted water-soluble prodrugs of selective COX-2 inhibitory drugs having a sulfonamide moiety. Parecoxib converts to the substantially water-insoluble selective COX-2 inhibitory drug valdecoxib following administration to a subject. Parecoxib also converts to valdecoxib upon exposure to water, for example upon dissolution in water.

Parecoxib, having the structural formula (I) below, itself shows weak in vitro inhibitory activity against both COX-1 and COX-2, while valdecoxib (II) has strong inhibitory activity against COX-2 but is a weak inhibitor of COX-1.

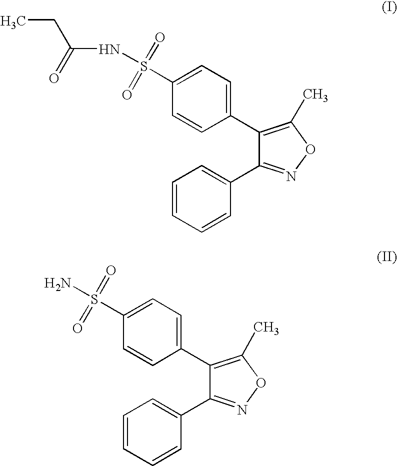

Above-cited U.S. Pat. No. 5,932,598 also discloses comparably N-substituted prodrugs of other selective COX-2 inhibitors having a sulfonamide moiety. For example, the compound N-[[4-[5-(4-methylphenyl)-3-(trifluoromethyl)-1H-pyrazol-1-yl]phenyl]sulfonyl]acetamide (III) and its sodium salt are contemplated therein to be useful as prodrugs of the selective COX-2 inhibitory drug celecoxib (IV).

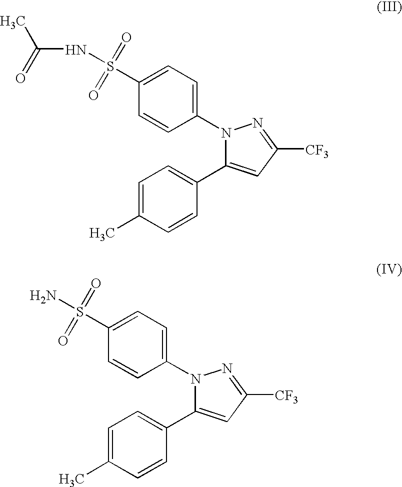

Because of the high water solubility of parecoxib, particularly of salts of parecoxib such as the sodium salt, by comparison with most selective COX-2 inhibitory drugs such as celecoxib and valdecoxib, the prodrug parecoxib has been proposed for parenteral use. See Talley et al. (2000), J. Med. Chem. 43, 1661-1663.

Above-cited U.S. Pat. No. 5,932,598 indicates that a preferred method of treating inflammation is administration of the water-soluble compounds disclosed therein via injection. However, the above-cited patent further discloses that the compounds disclosed therein, or a composition comprising such a compound, may be administered orally, and that for oral administration the composition may be in the form of, for example, a tablet, hard or soft capsule, lozenge, dispensable powder, suspension or liquid.

The tendency of parecoxib to convert rapidly to insoluble valdecoxib upon exposure to water has hitherto limited any interest in oral administration of parecoxib or in developing a practical oral dosage form of parecoxib.

IV administration is, for many classes of people suffering or at risk of such disorders, inconvenient and unpleasant, especially where self-administration is desired. Oral administration is generally much more convenient and conducive to a higher degree of patient compliance. It would be a further advantage if fast onset of therapeutic effect, especially for treatment of acute pain, were achievable by such administration.

### SUMMARY OF THE INVENTION

There is now provided a novel compound having the structural formula (V)

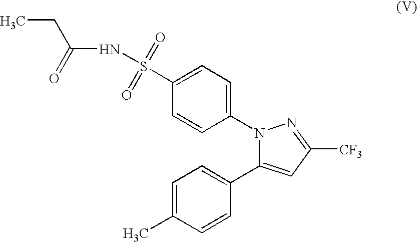

namely, N-[[4-[5-(4-methylphenyl)-3-(trifluoromethyl)-1H-pyrazol-1-yl]phenyl]-sulfonyl]propanamide, alternatively named 4-[5-(4-methylphenyl)-3-(trifluoromethyl)-1H-pyrazol-1-yl]-N-propionylbenzenesulfonamide. Further, there are provided pharmaceutically acceptable salts of (V), including the sodium salt (VI)

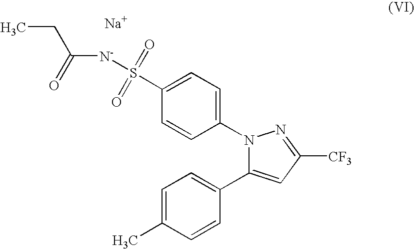

also known herein as “compound Z”.

Compound (V) and its salts are useful prodrugs of celecoxib, that can be administered to a subject by any suitable route, including parenterally (e.g., intravenously, intramuscularly, subcutaneously or intradermally), topically, transdermally, intraocularly, rectally or orally, for treatment or prophylaxis of a COX-2 mediated condition. However, it has unexpectedly been discovered, as described more fully hereinbelow, that when such prodrugs are administered orally, a surprisingly high blood plasma concentration of celecoxib is obtained within a short time interval following administration, consistent with a rapid onset therapeutic effect as strongly desired in the art.

Water-soluble salts of compound (V) are preferred, but present a problem in that, in presence of water, compound (V) and its salts tend to degrade rapidly to celecoxib. Accordingly, there is still further provided a pharmaceutical composition comprising in a unit dose thereof a therapeutically effective amount in total of at least one compound selected from N-[[4-[5-(4-methylphenyl)-3-(trifluoromethyl)-1H-pyrazol-1-yl]phenyl]-sulfonyl]propanamide and pharmaceutically acceptable salts thereof, the composition being orally deliverable and substantially free of water and having means for inhibiting degradation of said at least one compound to celecoxib prior to oral administration.

There is still further provided an article of manufacture comprising a substantially water-impermeable package, having contained therein a single unit dose of an orally deliverable pharmaceutical composition that is substantially free of water and comprises a therapeutically effective amount in total of at least one compound selected from N-[[4-[5-(4-methylphenyl)-3-(trifluoromethyl)-1H-pyrazol-1-yl]phenyl]sulfonyl]-propanamide and pharmaceutically acceptable salts thereof.

“Orally deliverable” herein means that the composition, either (a) as provided above, i.e., substantially free of water, or (b) following dispersion and/or dissolution of the composition in a pharmaceutically acceptable aqueous vehicle, is suitable for oral administration to a subject.

There is still further provided a method of treating or preventing a COX-2 mediated disorder in a subject, the method comprising (a) dissolving, in a pharmaceutically acceptable aqueous vehicle, at least one unit dose of a pharmaceutical composition that is substantially free of water and comprises a therapeutically effective amount in total of at least one compound selected from N-[[4-[5-(4-methylphenyl)-3-(trifluoromethyl)-1H-pyrazol-1-yl]phenyl]sulfonyl]propanamide and pharmaceutically acceptable salts thereof, to form a solution, and (b) orally administering the solution to the subject before substantial precipitation of insoluble matter occurs in the solution.

### BRIEF DESCRIPTION OF THE DRAWINGS

FIG. 1 presents data from a pharmacokinetic study in dogs, showing mean blood plasma concentrations of celecoxib from 0 to 24 hours following oral administration of (a) celecoxib formulated as a commercial capsule; (b) celecoxib in suspension in apple juice; and (c) celecoxib prodrug compound Z as defined above in aqueous solution; all in an amount equivalent to 200 mg celecoxib.

### DETAILED DESCRIPTION OF THE INVENTION

N-[[4-[5-(4-methylphenyl)-3-(trifluoromethyl)-1H-pyrazol-1-yl]phenyl]-sulfonyl]propanamide can illustratively be prepared, using celecoxib as a starting material, by the method described in Example 1 below. Salts of N-[[4-[5-(4-methylphenyl)-3-(trifluoromethyl)-1H-pyrazol-1-yl]phenyl]sulfonyl]propanamide can be prepared by reacting N-[[4-[5-(4-methylphenyl)-3-(trifluoromethyl)-1H-pyrazol-1-yl]phenyl]sulfonyl]propanamide with a suitable base, as illustrated by synthesis of the sodium salt in Example 2 below.

Pharmaceutically acceptable salts of N-[[4-[5-(4-methylphenyl)-3-(trifluoromethyl)-1H-pyrazol-1-yl]phenyl]sulfonyl]propanamide include, without limitation, metal salts, ammonium salts and organic ammonium salts. Suitable metal salts are alkali metal salts, including lithium, potassium and sodium salts, alkaline earth metal salts, including magnesium and calcium salts, and certain other physiologically acceptable metal salts, including aluminum and zinc salts. Presently preferred are alkali metal salts, particularly potassium and sodium salts, most particularly the sodium salt (VI). Suitable organic ammonium salts illustratively include diethylamine, diethanolamine, ethylenediamine, N,N′-dibenzylethylenediamine, tromethamine, procaine, chloroprocaine, choline and meglumine salts. Water-soluble salts are preferred, in particular those having solubility in water of at least about 10 mg/ml at room temperature.

Exposure to moisture of a composition containing N-[[4-[5-(4-methylphenyl)-3-(trifluoromethyl)-1H-pyrazol-1-yl]phenyl]sulfonyl]propanamide or a salt thereof tends to cause significant conversion to celecoxib. In such circumstances the composition remains therapeutically effective, celecoxib being the active drug for which N-[[4-[5-(4-methylphenyl)-3-(trifluoromethyl)-1H-pyrazol-1-yl]phenyl]-sulfonyl]propanamide is a prodrug, but certain benefits according to the present invention, in particular the benefits of rapid attainment of therapeutic blood plasma concentration, and consequent rapid onset of therapeutic effect, would tend to be reduced by such exposure.

In one embodiment, therefore, the invention provides a pharmaceutical composition that is substantially free of water, i.e., a dry composition. The term “substantially free of water” in the present context means that the amount of water present in the composition and available for reaction with the prodrug is sufficiently low that the composition exhibits acceptable chemical stability of the prodrug for at least about 30 days, preferably at least about 6 months, most preferably at least about 2 years, when stored at room temperature (about 20-25° C.) in a sealed water-impermeable container. “Acceptable chemical stability” herein means that the composition, following the defined time period (e.g., about 30 days, about 6 months or about 2 years), passes a standard test for chemical purity of the prodrug, for example as may be required for approval by a regulatory authority. An example of such a test is the “5% total, 1% single impurity rule”, whereby a preparation of a candidate drug or prodrug must contain not more than 5% total impurities, and not more than 1% of any single impurity.

Typically, a sufficiently low water content in the composition to provide acceptable chemical stability of N-[[4-[5-(4-methylphenyl)-3-(trifluoromethyl)-1H-pyrazol-1-yl]phenyl]sulfonyl]propanamide is less than about 5%, preferably less than about 2%, more preferably less than about 1%, by weight.

In this embodiment, the composition comprises in each unit dose thereof a therapeutically effective amount in total of at least one compound selected from N-[[4-[5-(4-methylphenyl)-3-(trifluoromethyl)-1H-pyrazol-1-yl]phenyl]sulfonyl]propanamide and pharmaceutically acceptable salts thereof. A “unit dose” herein means a portion of a pharmaceutical composition that contains an amount of the agent suitable for a single oral administration to provide a therapeutic effect. Typically one unit dose, or a small plurality (up to about 4) of unit doses, provides a sufficient amount of the agent to result in the desired effect. In this regard, when the terms “therapeutic effect”, “therapeutically effective” and “therapeutic agent” are applied herein to a prodrug, for example N-[[4-[5-(4-methylphenyl)-3-(trifluoromethyl)-1H-pyrazol-1-yl]phenyl]sulfonyl]propanamide or a water-soluble salt thereof, it will be understood that these terms are being used in the broad sense applicable to a prodrug which is converted to a therapeutically active compound. It will further be understood in this context that “therapeutic” embraces prophylactic.

A unit dose of a composition of the invention comprises an amount of the prodrug that is equivalent to, or that theoretically generates upon 100% conversion, an amount of celecoxib that is known in the literature to be therapeutically effective. For example, a therapeutically effective amount of compound Z is an amount equivalent to about 10 to about 1000 mg, more typically about 50 to about 400 mg, preferably about 100 to about 200 mg, for example 100 mg or 200 mg, celecoxib.

In the present embodiment, the dry composition has means for inhibiting conversion of N-[[4-[5-(4-methylphenyl)-3-(trifluoromethyl)-1H-pyrazol-1-yl]phenyl]-sulfonyl]propanamide or salt thereof to celecoxib prior to dissolution in an aqueous vehicle. Such means can operate to inhibit the conversion in one or more of a variety of ways including those indicated immediately below. All such means, as present in association with a composition as herein provided, are embraced by the present invention.

An example of means for inhibiting conversion of N-[[4-[5-(4-methylphenyl)-3-(trifluoromethyl)-1H-pyrazol-1-yl]phenyl]sulfonyl]propanamide or salt thereof to celecoxib in a dry composition of the invention is a means for substantially preventing exposure of the composition to water, including atmospheric humidity, during storage and transport. Exposure to water can be substantially prevented, for example, by enclosing the composition in a sealed and substantially water-impermeable package or container. Alternatively or in addition, the composition can be coated with a substantially water-impermeable coating material, e.g., an ethylcellulose-based coating material. Individual solid particles or granules of the composition, or larger beads or whole tablets of the composition, can be so coated. If used, a coating should be selected to be readily degradable in the gastrointestinal tract, so that the benefits of rapid absorption of the drug or prodrug are not cancelled out by delay in release of the drug or prodrug from the ingested composition.

A further example of means for inhibiting conversion of N-[[4-[5-(4-methylphenyl)-3-(trifluoromethyl)-1H-pyrazol-1-yl]phenyl]sulfonyl]propanamide or salt thereof to celecoxib in a dry composition of the invention is to formulate the composition in such a way as to avoid or minimize contact of the prodrug with any excipient other than water that would otherwise promote such conversion. For example, in one embodiment no such excipient is present in the composition. In another embodiment a barrier layer is present between the prodrug and any such excipient present.

Illustratively, certain saccharides, for example mannitol, that can be useful excipients in a composition of the invention, tend to promote conversion of parecoxib to valdecoxib in a dry composition where such an excipient is in intimate contact with the parecoxib. It is contemplated that such saccharides similarly promote conversion of N-[[4-[5-(4-methylphenyl)-3-(trifluoromethyl)-1H-pyrazol-1-yl]phenyl]sulfonyl]-propanamide or salts thereof to celecoxib. By pre-coating or encapsulating at least one of the excipient and the prodrug with a material that minimizes contact between them, such conversion can be inhibited.

Other means for inhibiting conversion of N-[[4-[5-(4-methylphenyl)-3-(trifluoromethyl)-1H-pyrazol-1-yl]phenyl]sulfonyl]propanamide or salt thereof to celecoxib in a dry composition of the invention will be apparent to those of skill in the art.

The dry composition of the invention is preferably substantially soluble in a pharmaceutically acceptable aqueous vehicle to form an orally deliverable solution. The term “substantially soluble” means that a unit dose of the composition dissolves in a volume of the aqueous vehicle not greater than about 100 ml, preferably not greater than about 50 ml, with no visually observable insoluble residue, except optionally for slight cloudiness arising only from excipient ingredients of the composition or of the aqueous vehicle.

Any pharmaceutically acceptable aqueous liquid is suitable as the vehicle or medium for dissolution of the composition. Water, for example tap water or bottled water, is particularly suitable. Alternatively, sweetened, flavored and/or carbonated beverages such as sugar solutions, fruit juices, sodas, infusions (e.g., teas), extracts (e.g., beef extract, malt extract, yeast extract) etc. can be used.

A composition of the invention can consist essentially of N-[[4-[5-(4-methylphenyl)-3-(trifluoromethyl)-1H-pyrazol-1-yl]phenyl]sulfonyl]propanamide or a water-soluble salt thereof, but optionally further comprises additional ingredients, for example pharmaceutically acceptable excipients. Such additional ingredients are preferably selected, and present in such amounts as, to be chemically compatible with the prodrug, in particular not to promote conversion of the prodrug to celecoxib in substantial absence of water. If a desired excipient is found to promote such conversion, a composition containing that excipient should be formulated with a barrier layer to avoid or minimize contact between the excipient and the prodrug as described above.

Examples of excipients that can be included in a composition of the invention are excipients that facilitate preparation of the composition, for example by processes hereinafter described. Such excipients include without limitation pharmaceutically acceptable bulking agents, buffering agents, anti-caking agents, etc.

Further examples of excipients that can be included in a composition of the invention are agents to enhance organoleptic properties upon dissolution of the composition. It has been found that parecoxib, specifically parecoxib sodium, has an unpleasantly bitter taste, and it is contemplated that N-[[4-[5-(4-methylphenyl)-3-(trifluoromethyl)-1H-pyrazol-1-yl]phenyl]sulfonyl]propanamide and salts thereof are similarly unpleasantly bitter to taste. Accordingly, in a preferred embodiment there is included in the composition at least one organoleptic-enhancing agent selected from sweeteners, flavoring agents and taste modulators. Suitable sweeteners include without limitation soluble sugars such as dextrose, fructose, sucrose and mannitol, and synthetic sweeteners such as saccharin, cyclamic acid, acesulfame, aspartame, neotame and salts thereof. Suitable natural or synthetic flavoring agents can be selected from standard reference books, for example Fenaroli's Handbook of Flavor Ingredients, 3rd edition (1995). Non-limiting examples of suitable natural flavors, some of which can readily be simulated with synthetic agents or combinations thereof, include almond, anise, apple, apricot, bergamot, blackberry, blackcurrant, blueberry, cacao, caramel, cherry, cinnamon, clove, coffee, coriander, cranberry, cumin, dill, eucalyptus, fennel, fig, ginger, grape, grapefruit, guava, hop, lemon, licorice, lime, malt, mandarin, molasses, nutmeg, orange, peach, pear, peppermint, raspberry, rose, spearmint, strawberry, tangerine, tea, vanilla, wintergreen, etc. Taste modulators are agents that affect a subject's perception of taste and include anesthetic agents.

Preferred excipients are those that dissolve completely in the aqueous vehicle. Accessory excipients can optionally be included to enhance dissolution of other ingredients; such accessory excipients include pharmaceutically acceptable wetting agents, cyclodextrins, etc.

The dry composition can be in any suitable form, but is preferably in a rapidly dissolving form, for example a powder (e.g., a powder prepared by lyophilization as hereinafter described) or a rapidly disintegrating tablet. Optionally an effervescent agent, for example a bicarbonate salt such as sodium bicarbonate, can be included to accelerate dissolution and to provide organoleptic benefits of effervescence.

A powder composition of the invention preferably has sufficient porosity to permit rapid dissolution of the therapeutic agent upon addition to an aqueous vehicle. A high degree of porosity is obtainable by using a lyophilization process to prepare the powder as described hereinbelow.

In an illustrative process, compound Z and a buffering agent, for example dibasic sodium phosphate heptahydrate, are dissolved in water to form an aqueous solution. Compound Z and the buffering agent are present in the solution at concentrations relative to each other consistent with the desired relative concentrations of these ingredients in the final composition. Absolute concentrations of these ingredients are not critical; however, in the interest of process efficiency it is generally preferred that the concentration of compound Z be as high as can be conveniently prepared without risking exceeding the limit of solubility. Other formulation ingredients can be added in this step if desired. Order of addition is not critical but it is strongly preferred to add the compound Z last to ensure rapid and complete dissolution, and to minimize the duration of exposure of the prodrug to water.

The solution is metered into one or more lyophilization containers, e.g., vials. Each container receives a measured volume of solution having a desired dosage amount of compound Z. Stoppers having an opening to allow sublimation to occur are placed on the containers. The stoppered containers are then placed in a lyophilization chamber and the contents of the containers are lyophilized under vacuum. On completion of the lyophilization cycle, the vacuum is released and temperature is permitted to return to room temperature. The containers are then sealed to prevent reabsorption of moisture from the atmosphere.

Discrete dosage forms such as tablets and capsules suitable for oral administration of N-[[4-[5-(4-methylphenyl)-3-(trifluoromethyl)-1H-pyrazol-1-yl]phenyl]sulfonyl]propanamide or a salt thereof can be prepared by methods known in the art. Methods that minimize amount and/or duration of water contact with the prodrug are preferred.

In another embodiment, the invention provides an article of manufacture comprising a substantially water-impermeable package having contained therein a single unit dose of an orally deliverable pharmaceutical composition that is substantially free of water and comprises a therapeutically effective amount in total of at least one compound selected from N-[[4-[5-(4-methylphenyl)-3-(trifluoromethyl)-1H-pyrazol-1-yl]phenyl]-sulfonyl]propanamide and pharmaceutically acceptable salts thereof. Preferably the composition is substantially soluble in a pharmaceutically acceptable aqueous vehicle to form an orally deliverable solution.

“Substantially water-impermeable” herein means that the package, when stored under normal atmospheric conditions, is sufficiently resistant to entry of moisture during a storage period of at least about 30 days, preferably at least about 6 months and more preferably at least about 2 years, such that the composition remains substantially free of water as defined herein.

Suitable packaging materials include without limitation glass, polypropylene, aluminum, etc. The package must be sealed against entry of moisture through any opening or seam. Because the package contains only a single unit dose of the composition, it does not have to be resealed after use.

Embraced by the above description is an article of manufacture comprising a plurality of conjoined substantially water-impermeable packages, each having contained therein a single unit dose of a composition of the invention. For example, rapidly water-dispersible (e.g., effervescent) unit-dose tablets can be individually packaged in a plurality of water-impermeable compartments of a conventional foil pack or blister pack.

In yet another embodiment, a method of treating or preventing a COX-2 mediated disorder in a subject is provided. The method comprises (a) dissolving, in a pharmaceutically acceptable aqueous vehicle, at least one unit dose of a pharmaceutical composition that is substantially free of water and comprises a therapeutically effective amount in total of at least one compound selected from N-[[4-[5-(4-methylphenyl)-3-(trifluoromethyl)-1H-pyrazol-1-yl]phenyl]sulfonyl]propanamide and pharmaceutically acceptable salts thereof, to form a solution, and (b) orally administering the solution to the subject before substantial precipitation of insoluble matter occurs in the solution.

The aqueous vehicle can be any pharmaceutically acceptable aqueous liquid, including those indicated hereinabove. Optionally, the aqueous vehicle can contain one or more ingredients such as sweeteners or flavoring agents to counteract the unpleasant taste of the prodrug, whether or not the dry composition comprises such ingredients.

Any convenient volume of the aqueous liquid can be used as the vehicle for oral administration of a unit dose of the composition. Typically a volume not greater than about 100 ml is preferred, and more preferably the volume is not greater than about 50 ml.

Where the dry composition is in the form of a powder, for example a lyophilized powder, it is generally most convenient to add the aqueous liquid to the container in which the powder is packaged. For this purpose, it is therefore preferred that the container be large enough to accommodate a suitable volume of liquid wherein, upon opening the container, the composition can be dissolved prior to administration.

Where the dry composition is in the form of a discrete dosage form, illustratively a tablet, one or more tablets can be added to a suitable volume of aqueous liquid in a drinking vessel, wherein the composition is dissolved prior to administration.

Agitation or stirring of the container or vessel wherein dissolution occurs may be desirable to accelerate the process of dissolution. Preferred compositions of the invention require only mild or no agitation or stirring.

The resulting solution is preferably administered as soon as dissolution is complete. Delay in administration can result in precipitation of insoluble celecoxib in the solution, thereby reducing the benefits obtainable by the method of the invention. Typically oral administration should occur less than about 15 minutes, preferably less than about 5 minutes, after preparation of the solution.

Compositions of the invention are useful in treatment and prevention of a very wide range of disorders mediated by COX-2, including but not restricted to disorders characterized by inflammation, pain and/or fever. Such compositions are especially useful as anti-inflammatory agents, such as in treatment of arthritis, with the additional benefit of having significantly less harmful side effects than compositions of conventional NSAIDs that lack selectivity for COX-2 over COX-1. In particular, compositions of the invention have reduced potential for gastrointestinal toxicity and gastrointestinal irritation, including upper gastrointestinal ulceration and bleeding, by comparison with compositions of conventional NSAIDs. Thus compositions of the invention are particularly useful as an alternative to conventional NSAIDs where such NSAIDs are contraindicated, for example in patients with peptic ulcers, gastritis, regional enteritis, ulcerative colitis, diverticulitis or with a recurrent history of gastrointestinal lesions; gastrointestinal bleeding, coagulation disorders including anemia such as hypoprothrombinemia, hemophilia or other bleeding problems; kidney disease; or in patients prior to surgery or patients taking anticoagulants.

Contemplated compositions are useful to treat a variety of arthritic disorders, including but not limited to rheumatoid arthritis, spondyloarthropathies, gouty arthritis, osteoarthritis, systemic lupus erythematosus and juvenile arthritis.

Such compositions are useful in treatment of asthma, bronchitis, menstrual cramps, preterm labor, tendonitis, bursitis, allergic neuritis, cytomegalovirus infection, apoptosis including HIV-induced apoptosis, lumbago, liver disease including hepatitis, skin-related conditions such as psoriasis, eczema, acne, burns, dermatitis and ultraviolet radiation damage including sunburn, and post-operative inflammation including that following ophthalmic surgery such as cataract surgery or refractive surgery.

Such compositions are useful to treat gastrointestinal conditions such as inflammatory bowel disease, Crohn's disease, gastritis, irritable bowel syndrome and ulcerative colitis.

Such compositions are useful in treating inflammation in such diseases as migraine headaches, periarteritis nodosa, thyroiditis, aplastic anemia, Hodgkin's disease, scleroderma, rheumatic fever, type I diabetes, neuromuscular junction disease including myasthenia gravis, white matter disease including multiple sclerosis, sarcoidosis, nephrotic syndrome, Behcet's syndrome, polymyositis, gingivitis, nephritis, hypersensitivity, swelling occurring after injury including brain edema, myocardial ischemia, and the like.

Such compositions are useful in treatment of ophthalmic disorders, including without limitation inflammatory disorders such as endophthalmitis, episcleritis, retinitis, iriditis, cyclitis, choroiditis, keratitis, conjunctivitis and blepharitis, inflammatory disorders of more than one part of the eye, e.g., retinochoroiditis, iridocyclitis, iridocyclochoroiditis (also known as uveitis), keratoconjunctivitis, blepharoconjunctivitis, etc.; other COX-2 mediated retinopathies including diabetic retinopathy; ocular photophobia; acute trauma of any tissue of the eye including postsurgical trauma, e.g., following cataract or corneal transplant surgery; postsurgical ocular inflammation; intraoperative miosis; corneal graft rejection; ocular, for example retinal, neovascularization including that following injury or infection; macular degeneration; cystoid macular edema; retrolental fibroplasia; neovascular glaucoma; and ocular pain.

Such compositions are useful in treatment of pulmonary inflammation, such as that associated with viral infections and cystic fibrosis, and in bone resorption such as that associated with osteoporosis.

Such compositions are useful for treatment of certain central nervous system disorders, such as cortical dementias including Alzheimer's disease, neurodegeneration, and central nervous system damage resulting from stroke, ischemia and trauma. The term “treatment” in the present context includes partial or total inhibition of dementias, including Alzheimer's disease, vascular dementia, multi-infarct dementia, pre-senile dementia, alcoholic dementia and senile dementia.

Such compositions are useful in treatment of allergic rhinitis, respiratory distress syndrome, endotoxin shock syndrome and liver disease.

Such compositions are useful in treatment of pain, including but not limited to postoperative pain, dental pain, muscular pain, and pain resulting from cancer. For example, such compositions are useful for relief of pain, fever and inflammation in a variety of conditions including rheumatic fever, influenza and other viral infections including common cold, low back and neck pain, dysmenorrhea, headache, toothache, sprains and strains, myositis, neuralgia, synovitis, arthritis, including rheumatoid arthritis, degenerative joint diseases (osteoarthritis), gout and ankylosing spondylitis, bursitis, burns, and trauma following surgical and dental procedures.

Such compositions are useful for treating and preventing inflammation-related cardiovascular disorders, including vascular diseases, coronary artery disease, aneurysm, vascular rejection, arteriosclerosis, atherosclerosis including cardiac transplant atherosclerosis, myocardial infarction, embolism, stroke, thrombosis including venous thrombosis, angina including unstable angina, coronary plaque inflammation, bacterial-induced inflammation including Chlamydia-induced inflammation, viral induced inflammation, and inflammation associated with surgical procedures such as vascular grafting including coronary artery bypass surgery, revascularization procedures including angioplasty, stent placement, endarterectomy, or other invasive procedures involving arteries, veins and capillaries.

Such compositions are useful in treatment of angiogenesis-related disorders in a subject, for example to inhibit tumor angiogenesis. Such compositions are useful in treatment of neoplasia, including metastasis; ophthalmological conditions such as corneal graft rejection, ocular neovascularization, retinal neovascularization including neovascularization following injury or infection, diabetic retinopathy, macular degeneration, retrolental fibroplasia and neovascular glaucoma; ulcerative diseases such as gastric ulcer; pathological, but non-malignant, conditions such as hemangiomas, including infantile hemangiomas, angiofibroma of the nasopharynx and avascular necrosis of bone; and disorders of the female reproductive system such as endometriosis.

Such compositions are useful in prevention and treatment of benign and malignant tumors and neoplasia including cancer, such as colorectal cancer, brain cancer, bone cancer, epithelial cell-derived neoplasia (epithelial carcinoma) such as basal cell carcinoma, adenocarcinoma, gastrointestinal cancer such as lip cancer, mouth cancer, esophageal cancer, small bowel cancer, stomach cancer, colon cancer, liver cancer, bladder cancer, pancreas cancer, ovary cancer, cervical cancer, lung cancer, breast cancer, skin cancer such as squamous cell and basal cell cancers, prostate cancer, renal cell carcinoma, and other known cancers that effect epithelial cells throughout the body. Neoplasias for which compositions of the invention are contemplated to be particularly useful are gastrointestinal cancer, Barrett's esophagus, liver cancer, bladder cancer, pancreatic cancer, ovarian cancer, prostate cancer, cervical cancer, lung cancer, breast cancer and skin cancer. Such compositions can also be used to treat fibrosis that occurs with radiation therapy. Such compositions can be used to treat subjects having adenomatous polyps, including those with familial adenomatous polyposis (FAP). Additionally, such compositions can be used to prevent polyps from forming in subjects at risk of FAP.

Such compositions inhibit prostanoid-induced smooth muscle contraction by inhibiting synthesis of contractile prostanoids and hence can be of use in treatment of dysmenorrhea, premature labor, asthma and eosinophil-related disorders. They also can be of use for decreasing bone loss particularly in postmenopausal women (i.e., treatment of osteoporosis), and for treatment of glaucoma.

Preferred uses for compositions of the present invention are for treatment of rheumatoid arthritis and osteoarthritis, for pain management generally (particularly post-oral surgery pain, post-general surgery pain, post-orthopedic surgery pain, and acute flares of osteoarthritis), for prevention and treatment of headache and migraine, for treatment of Alzheimer's disease, and for colon cancer chemoprevention.

Because of the rapid onset of therapeutic effect that can be exhibited by compositions of the invention, these compositions have particular advantages over prior orally deliverable compositions of selective COX-2 inhibitory drugs for treatment of acute COX-2 mediated disorders, especially for relief of pain, for example in headache, including sinus headache and migraine.

Besides being useful for human treatment, compositions of the invention are useful for veterinary treatment of companion animals, exotic animals, farm animals, and the like, particularly mammals. More particularly, compositions of the invention are useful for treatment of COX-2 mediated disorders in horses, dogs and cats.

The dosage regimen to prevent, give relief from, or ameliorate the condition or disorder preferably corresponds to once-a-day or twice-a-day treatment, but can be modified in accordance with a variety of factors. These include the type, age, weight, sex, diet and medical condition of the subject and the nature and severity of the disorder. Thus, the dosage regimen actually employed can vary widely and can therefore deviate from the preferred dosage regimens set forth above.

Initial treatment can begin with a dose regimen as indicated above. Treatment is generally continued as necessary over a period of several weeks to several months or years until the condition or disorder has been controlled or eliminated. Subjects undergoing treatment with a composition of the invention can be routinely monitored by any of the methods well known in the art to determine effectiveness of therapy. Continuous analysis of data from such monitoring permits modification of the treatment regimen during therapy so that optimally effective doses are administered at any point in time, and so that the duration of treatment can be determined. In this way, the treatment regimen and dosing schedule can be rationally modified over the course of therapy so that the lowest amount of the composition exhibiting satisfactory effectiveness is administered, and so that administration is continued only for so long as is necessary to successfully treat the condition or disorder.

Preferably, the N-[[4-[5-(4-methylphenyl)-3-(trifluoromethyl)-1H-pyrazol-1-yl]phenyl]sulfonyl]propanamide or salt thereof, for example the sodium salt, is administered in a daily dosage amount equivalent to about 10 mg to about 1000 mg celecoxib. More preferred daily dosage amounts are equivalent to about 50 mg to about 400 mg, for example about 100 mg or about 200 mg, celecoxib.

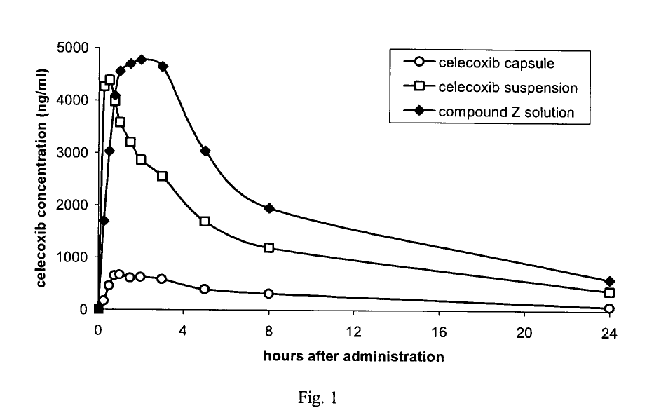

In an especially surprising finding, illustrated in FIG. 1, so rapid and complete is the in vivo conversion of N-[[4-[5-(4-methylphenyl)-3-(trifluoromethyl)-1H-pyrazol-1-yl]phenyl]sulfonyl]propanamide or a salt thereof to celecoxib that oral administration of the prodrug provides an early peak of blood plasma concentration of celecoxib that is at least comparable with that provided by oral administration of celecoxib itself at equal dose in an immediate release form.

Therapeutic methods of the present invention further include combination therapies of a composition of the invention with one or more drugs selected from opioids and other analgesics, including narcotic analgesics, Mu receptor antagonists, Kappa receptor antagonists, non-narcotic (i.e. non-addictive) analgesics, monoamine uptake inhibitors, adenosine regulating agents, cannabinoid derivatives, Substance P antagonists, neurokinin-1 receptor antagonists and sodium channel blockers, among others. Preferred combination therapies comprise use of a composition of the invention with one or more compounds selected from aceclofenac, acemetacin, ε-acetamidocaproic acid, acetaminophen, acetaminosalol, acetanilide, acetylsalicylsalicylic acid, S-adenosylmethionine, alclofenac, alfentanil, allylprodine, alminoprofen, aloxiprin, alphaprodine, aluminum bis(acetylsalicylate), amfenac, aminochlorthenoxazin, 3-amino-4-hydroxybutyric acid, 2-amino-4-picoline, aminopropylon, aminopyrine, amixetrine, ammonium salicylate, ampiroxicam, amtolmetin guacil, anileridine, antipyrine, antipyrine salicylate, antrafenine, apazone, aspirin, balsalazide, bendazac, benorylate, benoxaprofen, benzpiperylon, benzydamine, benzylmorphine, berberine, bermoprofen, bezitramide, α-bisabolol, bromfenac, p-bromoacetanilide, 5-bromosalicylic acid acetate, bromosaligenin, bucetin, bucloxic acid, bucolome, bufexamac, bumadizon, buprenorphine, butacetin, butibufen, butorphanol, calcium acetylsalicylate, carbamazepine, carbiphene, carprofen, carsalam, chlorobutanol, chlorthenoxazin, choline salicylate, cinchophen, cinmetacin, ciramadol, clidanac, clometacin, clonitazene, clonixin, clopirac, clove, codeine, codeine methyl bromide, codeine phosphate, codeine sulfate, cropropamide, crotethamide, desomorphine, dexoxadrol, dextromoramide, dezocine, diampromide, diclofenac, difenamizole, difenpiramide, diflunisal, dihydrocodeine, dihydrocodeinone enol acetate, dihydromorphine, dihydroxyaluminum acetylsalicylate, dimenoxadol, dimepheptanol, dimethylthiambutene, dioxaphetyl butyrate, dipipanone, dipyrocetyl, dipyrone, ditazol, droxicam, emorfazone, enfenamic acid, epirizole, eptazocine, etanercept, etersalate, ethenzamide, ethoheptazine, ethoxazene, ethylmethylthiambutene, ethylmorphine, etodolac, etofenamate, etonitazene, eugenol, felbinac, fenbufen, fenclozic acid, fendosal, fenoprofen, fentanyl, fentiazac, fepradinol, feprazone, floctafenine, flufenamic acid, flunoxaprofen, fluoresone, flupirtine, fluproquazone, flurbiprofen, fosfosal, gentisic acid, glafenine, glucametacin, glycol salicylate, guaiazulene, hydrocodone, hydromorphone, hydroxypethidine, ibufenac, ibuprofen, ibuproxam, imidazole salicylate, indomethacin, indoprofen, infliximab, interleukin-10, isofezolac, isoladol, isomethadone, isonixin, isoxepac, isoxicam, ketobemidone, ketoprofen, ketorolac, p-lactophenetide, lefetamine, levorphanol, lexipafant, lofentanil, lonazolac, lornoxicam, loxoprofen, lysine acetylsalicylate, magnesium acetylsalicylate, meclofenamic acid, mefenamic acid, meperidine, meptazinol, mesalamine, metazocine, methadone, methotrimeprazine, metiazinic acid, metofoline, metopon, mofebutazone, mofezolac, morazone, morphine, morphine hydrochloride, morphine sulfate, morpholine salicylate, myrophine, nabumetone, nalbuphine, 1-naphthyl salicylate, naproxen, narceine, nefopam, nicomorphine, nifenazone, niflumic acid, nimesulide, 5′-nitro-2′-propoxyacetanilide, norlevorphanol, normethadone, normorphine, norpipanone, olsalazine, opium, oxaceprol, oxametacine, oxaprozin, oxycodone, oxymorphone, oxyphenbutazone, papaveretum, paranyline, parsalmide, pentazocine, perisoxal, phenacetin, phenadoxone, phenazocine, phenazopyridine hydrochloride, phenocoll, phenoperidine, phenopyrazone, phenyl acetylsalicylate, phenylbutazone, phenyl salicylate, phenyramidol, piketoprofen, piminodine, pipebuzone, piperylone, pirazolac, piritramide, piroxicam, pirprofen, pranoprofen, proglumetacin, proheptazine, promedol, propacetamol, propiram, propoxyphene, propyphenazone, proquazone, protizinic acid, ramifenazone, remifentanil, rimazolium metilsulfate, salacetamide, salicin, salicylamide, salicylamide o-acetic acid, salicylsulfuric acid, salsalate, salverine, simetride, sodium salicylate, sufentanil, sulfasalazine, sulindac, superoxide dismutase, suprofen, suxibuzone, talniflumate, tenidap, tenoxicam, terofenamate, tetrandrine, thiazolinobutazone, tiaprofenic acid, tiaramide, tilidine, tinoridine, tolfenamic acid, tolmetin, tramadol, tropesin, viminol, xenbucin, ximoprofen, zaltoprofen, ziconotide and zomepirac (see The Merck Index, 13th Edition (2001), Therapeutic Category and Biological Activity Index, lists therein headed “Analgesic”, “Anti-inflammatory” and “Antipyretic”).

Particularly preferred combination therapies comprise use of a composition of the invention with an opioid compound, more particularly where the opioid compound is codeine, meperidine, morphine or a derivative thereof.

The drug being used in combination therapy with a composition of the invention can be administered by any route, including parenterally, orally, topically, etc. Where both the N-[[4-[5-(4-methylphenyl)-3-(trifluoromethyl)-1H-pyrazol-1-yl]phenyl]sulfonyl]propanamide or salt thereof and the drug to be administered in combination therewith are both delivered orally, they can be formulated separately or co-formulated in a composition of the invention. Where N-[[4-[5-(4-methylphenyl)-3-(trifluoromethyl)-1H-pyrazol-1-yl]phenyl]sulfonyl]propanamide or a salt thereof is co-formulated with a second drug, for example an opioid drug, the second drug can be formulated in immediate-release, rapid-onset, sustained-release or dual-release form.

In an embodiment of the invention, particularly where the COX-2 mediated condition is headache or migraine, the present composition is administered in combination therapy with a vasomodulator, preferably a xanthine derivative having vasomodulatory effect, more preferably an alkylxanthine compound.

Combination therapies wherein an alkylxanthine compound is co-administered with a composition as provided herein are embraced by the present embodiment of the invention whether or not the alkylxanthine is a vasomodulator and whether or not the therapeutic effectiveness of the combination is to any degree attributable to a vasomodulatory effect. The term “alkylxanthine” herein embraces xanthine derivatives having one or more C₁₋₄ alkyl, preferably methyl, substituents, and pharmaceutically acceptable salts of such xanthine derivatives. Dimethylxanthines and trimethylxanthines, including caffeine, theobromine and theophylline, are especially preferred. Most preferably, the alkylxanthine compound is caffeine.

The total and relative dosage amounts of N-[[4-[5-(4-methylphenyl)-3-(trifluoromethyl)-1H-pyrazol-1-yl]phenyl]sulfonyl]propanamide or salt thereof and of the vasomodulator or alkylxanthine are selected to be therapeutically and/or prophylactically effective for relief of pain associated with the headache or migraine. Suitable dosage amounts will depend on the particular vasomodulator or alkylxanthine selected. For example, in a combination therapy with a celecoxib prodrug and caffeine, typically the celecoxib prodrug will be administered in a daily dosage amount equivalent to about 50 mg to about 400 mg, preferably about 100 mg to about 200 mg, of celecoxib, and the caffeine in a daily dosage amount of about 1 mg to about 500 mg, preferably about 10 mg to about 400 mg, more preferably about 20 mg to about 300 mg.

The vasomodulator or alkylxanthine component of the combination therapy can be administered in any suitable dosage form by any suitable route, preferably orally. The vasomodulator or alkylxanthine can optionally be coformulated with N-[[4-[5-(4-methylphenyl)-3-(trifluoromethyl)-1H-pyrazol-1-yl]phenyl]sulfonyl]-propanamide in a single oral dosage form. Thus a composition of the invention optionally comprises both N-[[4-[5-(4-methylphenyl)-3-(trifluoromethyl)-1H-pyrazol-1-yl]phenyl]sulfonyl]-propanamide and a vasomodulator or alkylxanthine such as caffeine, in total and relative amounts consistent with the dosage amounts set out hereinabove. Alternatively, the N-[[4-[5-(4-methylphenyl)-3-(trifluoromethyl)-1H-pyrazol-1-yl]phenyl]sulfonyl]-propanamide can be present in a dry composition suitable for dissolution in an aqueous vehicle as provided herein, and the vasomodulator or alkylxanthine can be present in the aqueous vehicle. For example, a caffeinated beverage such as tea, coffee, or a caffeinated soda or sports beverage can be used as the vehicle for dissolution of a composition of the invention.

### EXAMPLES

The following examples illustrate an aspect of the present invention but are not to be construed as limitations.

### Example 1

Preparation of 4-[5-(4-methylphenyl)-3-(trifluoromethyl)-1H-pyrazol-1-yl]-N-propionylbenzenesulfonamide.

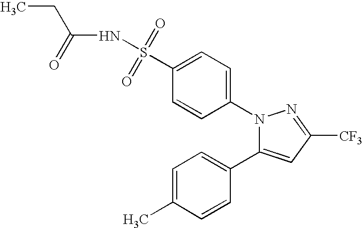

Celecoxib (4-[5-(4-methylphenyl)-3-(trifluoromethyl)-1H-pyrazol-1-yl]benzenesulfonamide) (0.2 mol, 76.3 g), tetrahydrofuran (300 ml), propionic anhydride (0.4 mol, 52.1 g), triethylamine (0.22 mol, 22.3 g), and 4-dimethylaminopyridine (0.02 mol, 2.44 g) were stirred at reflux for 4 h. The mixture was concentrated, dissolved in ethyl acetate and washed successively with hydrochloric acid (1N), brine, and water. After drying over magnesium sulfate and concentrating under high vacuum, the mixture was dissolved in ethanol and stirred for 4 h. A white solid was collected by filtration (79.1 g, 90.4%).

mp 88.3-96.7° C. Anal. Calculated for C₂₀H₁₈N₃SO₃F₃: C, 54.91; H, 4.15; N, 9.61. Found: C, 54.84; H, 4.23; N, 9.52. ¹H NMR (D₆-acetone): 11.6 (brs, 1H), 8.06 (d, 2H), 7.59 (d, 2H), 7.23 (s, 4H), 6.99 (s, 1H), 2.8 (m, 2H), 0.98 (t, 3H).

### Example 2

Preparation of 4-[5-(4-methylphenyl)-3-(trifluoromethyl)-1H-pyrazol-1-yl]-N-propionylbenzenesulfonamide, sodium salt (compound Z).

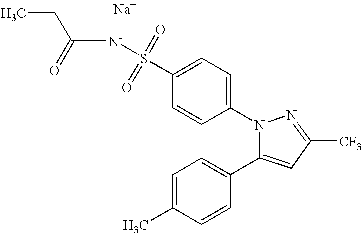

The compound prepared in Example 1 (4-[5-(4-methylphenyl)-3-(trifluoromethyl)-1H-pyrazol-1-yl]-N-propionylbenzenesulfonamide) (0.18 mol, 78.7 g) and ethanol (300 ml) were stirred at room temperature when sodium hydroxide (0.4936 N, 0.18 mol, 364.5 ml) was added. After 0.5 h, the mixture was concentrated, water (deionized, 300 ml) was added and the mixture was re-concentrated. This process was repeated, and the product, a white solid, was obtained after drying at 70° for 2 d. (81.7 g, 98.8%).

mp>300° C. Anal. Calculated for C₂₀H₁₇N₃SO₃F₃Na: C, 52.29; H, 3.73; N, 9.15. Found: C, 52.17; H, 3.72; N, 9.22.

### Example 3

Blood plasma concentration of celecoxib in beagle dogs was determined in a pharmacokinetic study using 6 healthy adult male subjects. Each subject received each of three treatments as detailed below. Treatments (a) and (b) were administered at an earlier time, in randomized sequence, than treatment (c), but to the same dogs. The treatments were:

(a) a single oral 200 mg dose of celecoxib in the form of a Celebrex® capsule;

(b) a single oral 200 mg dose of celecoxib in the form of a freshly prepared suspension in apple juice; and

(c) a single oral dose of compound Z in aqueous solution at a concentration of 24.1 mg/ml, equivalent to 20 mg/ml celecoxib, in an amount of 10 ml.

Each treatment was administered as a bolus dose by gastric intubation, followed by 10 ml water.

Celecoxib blood plasma concentration was determined using a validated high performance liquid chromatography (HPLC) procedure. The mean plasma concentration of celecoxib from 0 to 24 hours postdose is shown in FIG. 1. Calculated plasma pharmacokinetic parameters for celecoxib are given in Table 1.

TABLE 1
Pharmacokinetic parameters for celecoxib in plasma (mean ± s.d.)

| | | celecoxib apple | compound Z |
|---|---|---|---|
| parameter | celecoxib capsule | juice suspension | solution |
| Cₘₐₓ (ng/ml) | 852 ± 690 | 4602 ± 1305 | 5040 ± 1298 |
| Tₘₐₓ (hours) | 1.05 ± 1.10 | 0.33 ± 0.13 | 1.83 ± 0.68 |
| AUC (ng. hr/ml) | 6792 ± 5822 | 30635 ± 16590 | 55733 ± 32451 |

What is claimed is:

1. A compound having the formula 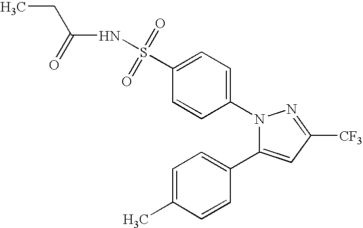
or a pharmaceutically acceptable salt thereof.

2. The compound of claim 1 that is the sodium salt of N-[[4-[5-(4-methylphenyl)-3-(trifluoromethyl)-1H-pyrazol-1-yl]phenyl]sulfonyl]propanamide.

3. A pharmaceutical composition comprising in a unit dose thereof a therapeutically effective amount in total of at least one compound selected from N-[[4-[5-(4-methylphenyl)-3-(trifluoromethyl)-1H-pyrazol-1-yl]phenyl]sulfonyl]propanamide and pharmaceutically acceptable salts thereof, the composition being orally deliverable and substantially free of water and having means for inhibiting degradation of said at least one compound to celecoxib prior to oral administration.

4. The composition of claim 3 wherein said at least one compound is the sodium salt of N-[[4-[5-(4-methylphenyl)-3-(trifluoromethyl)-1H-pyrazol-1-yl]phenyl]sulfonyl]-propanamide.

5. The composition of claim 3 wherein said therapeutically effective amount is equivalent to about 10 mg to about 1000 mg celecoxib.

6. The composition of claim 3 wherein said therapeutically effective amount is equivalent to about 50 mg to about 400 mg celecoxib.

7. The composition of claim 3 wherein the degradation inhibiting means comprises means for substantially preventing exposure of the composition to water.

8. The composition of claim 7 wherein the exposure preventing means comprises a sealed and substantially water-impermeable package or container.

9. The composition of claim 7 wherein the exposure preventing means comprises a substantially water-impermeable coating.

10. The composition of claim 3 wherein the degradation inhibiting means comprises a formulation of the composition having substantially no amount of any excipient that tends to promote such degradation when in intimate contact with said at least one compound.

11. The composition of claim 5 that comprises an excipient that tends to promote degradation of said at least one compound to celecoxib, wherein the degradation inhibiting means comprises a formulation of the composition having a barrier layer between said excipient and said at least one compound.

12. An article of manufacture comprising a substantially water-impermeable package, having contained therein a single unit dose of an orally deliverable pharmaceutical composition that is substantially free of water and comprises a therapeutically effective amount in total of at least one compound selected from N-[[4-[5-(4-methylphenyl)-3-(trifluoromethyl)-1H-pyrazol-1-yl]phenyl]-sulfonyl]propanamide and pharmaceutically acceptable salts thereof.

13. The article of claim 12 wherein said at least one compound is the sodium salt of N-[[4-[5-(4-methylphenyl)-3-(trifluoromethyl)-1H-pyrazol-1-yl]phenyl]sulfonyl]-propanamide.

14. The article of claim 12 wherein the composition is in a form of a powder and the package is a sealed container suitable when opened for addition of an aqueous vehicle wherein the composition can be dissolved.

15. The article of claim 12 wherein the composition is in a form of a tablet and the package is a foil pack or blister pack.

16. A method of treatment or prophylaxis of a COX-2 mediated condition in a subject, the method comprising administering a therapeutically effective amount in total of at least one compound selected from the group consisting of N-[[4-[5-(4-methylphenyl)-3-(trifluoromethyl)-1H-pyrazol-1-yl]phenyl]sulfonyl]propanamide and pharmaceutically acceptable salts thereof to the subject.

17. The method of claim 16 wherein said at least one compound is the sodium salt of N-[[4-[5-(4-methylphenyl)-3-(trifluoromethyl)-1H-pyrazol-1-yl]phenyl]sulfonyl]-propanamide.

18. A method of treating or preventing a COX-2 mediated disorder in a subject, the method comprising (a) dissolving, in a pharmaceutically acceptable aqueous vehicle, at least one unit dose of a pharmaceutical composition that is substantially free of water and comprises a therapeutically effective amount in total of at least one compound selected from N-[[4-[5-(4-methylphenyl)-3-(trifluoromethyl)-1H-pyrazol-1-yl]phenyl]sulfonyl]propanamide and pharmaceutically acceptable salts thereof, to form a solution, and (b) orally administering the solution to the subject before substantial precipitation of insoluble matter occurs in the solution.

19. The method of claim 18 wherein said at least one compound is the sodium salt of N-[[4-[5-(4-methylphenyl)-3-(trifluoromethyl)-1H-pyrazol-1-yl]phenyl]sulfonyl]-propanamide.
